SEQUENCE LISTING (14 sequences)

SEQ ID NO: 1 - protein, 5 aa, Zucchini yellow mosaic virus
  MISC_FEATURE (1)..(5): NIa-protease cleavage site
SVRLX

SEQ ID NO: 2 - DNA, 6 bp, Artificial
  Poly(A) addition signal
aataaa

SEQ ID NO: 3 - DNA, 27 bp, Zucchini yellow mosaic virus
tattcgtcgcaaccggaagttcagttc

SEQ ID NO: 4 - protein, 9 aa, Zucchini yellow mosaic virus
YSSQPEVQF

SEQ ID NO: 5 - protein, 27 aa, Artificial
  NcoI site was created between the N-terminal 2nd and 3rd amino acid of the HC-Pro coding sequence for insertion of a foreign gene
TATTCGTCGACCATGGAAGTTCAGTTC

SEQ ID NO: 6 - protein, 9 aa, Artificial
  NcoI site was created between the N-terminal 2nd and 3rd amino acid of the HC-Pro coding sequence for insertion of a foreign gene
YSSTMEVQF

SEQ ID NO: 7 - DNA, 36 bp, Artificial
  Coding sequence for a fragment of ZYMV-GFPHis
gaccactattcgtcgaccatggcatgcgggcccgtg

SEQ ID NO: 8 - protein, 12 aa, Artificial
  Fragment of ZYMV-GFPHis
DHYSSTMACGPV

SEQ ID NO: 9 - DNA, 72 bp, Artificial
  Coding sequence for a fragment of ZYMV-GFPHis
tacaagacgcgtggtaccccgcggcatcatcaccatcatcactccgtacggctccagtcatccatggaagtt

SEQ ID NO: 10 - protein, 24 aa, Artificial
  Fragment of ZYMV-GFPHis
YKTRGTPRHHHHHHSVRLQSSMEV

SEQ ID NO: 11 - DNA, 30 bp, Artificial
  Coding sequence for a fragment of ZYMV-Derp5
gaccactattcgtcgaccatggcatgcgat

SEQ ID NO: 12 - protein, 10 aa, Artificial
  Fragment of ZYMV-Derp5
DHYSSTMACD

SEQ ID NO: 13 - DNA, 63 bp, Artificial
  Coding sequence for a fragment of ZYMV-Derp5
gttggtaccccgcggcatcatcaccatcatcactccgtacggctccagtcatccatggaagtt

SEQ ID NO: 14 - protein, 21 aa, Artificial
  Fragment of ZYMV-Derp5
VGTPRHHHHHHSVRLQSSMEV

(19) United States
(12) Patent Application Publication
(10) Pub. No.: US 2005/0150014 A1
(43) Pub. Date: Jul. 7, 2005

(54) Process for producing dust mite allergen

(75) Inventors: Ching-Hsiang Hsu, Tainan County (CN); Wei-Chih Su, Tainan County (CN); Shyi-Dong Yeh, Taichung City (CN); Shih-Shun Lin, Taichung City (CN)

Correspondence Address: BANNER & WITCOFF, 1001 G STREET N W, SUITE 1100, WASHINGTON, DC, 20001, US

(73) Assignee: GenMont Biotech Inc., Tainan County (CN)

(21) Appl. No.: 10/750,887

(22) Filed: Jan. 5, 2004

(51) Int. Cl. A61K 39/00; A01H 1/00; C12N 15/82

(52) U.S. Cl. 800/288; 435/468; 424/184.1

(57) ABSTRACT

The present invention provides a process for producing dust mite allergen comprising the steps of: (a) constructing a vector that comprises a DNA sequence encoding the dust mite allergen operably linked to a plant-specific promoter; (b) transforming a plant cell or tissue with the vector of step (a); and (c) obtaining the dust mite allergen from the transgenic plant of step (b). A process for producing an antigenic composition and an antigenic composition are also provided.

### BACKGROUND OF THE INVENTION

1 Field of the invention

The invention mainly relates to a process for producing dust mite allergen.

2. Description of the Related Art

Allergy refers to an acquired potential to develop immunologically mediated adverse reaction to normally innocuous substances. Allergic reaction provokes symptoms such as itching, coughing, wheezing, sneezing, watery eyes, inflammation and fatigue. Many allergic diseases are due to several kinds of symptoms which are developed by sensitization to the antigen causing the diseases. In an allergic disease, an IgE antibody specific for an allergen (e.g., pollens and mite dust) in blood serum and tissue is produced, and when the antibody is exposed again to the antigen, the antibody reacts with the antigen in each tissue. It is normally believed that an allergic reaction includes an early specific immune response and a late inflammatory reaction. It is reported that an allergen mediates the early phase of allergy by stimulating high affinity immunoglobulin (IgE) receptors. Mast cells and basophils, when stimulated by allergens, will release histamine and cytokines. The cytokines released from mast cells and basophils then mediate the late phase of allergy by recruiting inflammatory cells.

It is reported that allergic diseases, such as bronchial asthma, childhood asthma, atopic dermatitis and the like, are mainly caused by allergens from mites living in house dust. Several kinds of proteins of mite allergens have been identified such as Der p 1, Der p 2 and Der p 5. Although only 60% of mite allergic children reacted to Der p 5, the IgE reactivity appeared to be stronger than that of Der p 1 and Der p 2 in Taiwan. Furthermore, among the various allergic diseases, the group of children with asthma has significant more reactivity than the group with rhinitis alone. Der p 5 is regarded as a clinically significant allergen in mite allergy.

Various approaches to the treatment and prevention of allergy were pursued. Feeding protein antigen to down-regulate systemic immune responses is a recognized method of inducing tolerance (Weiner H L. Oral tolerance:immune mechanisms and treatment of autoimmune diseases. Immunolo Today 1997;18:335-42). There has been interest in the potential of modulating autoinunune, inflammatory, and allergic disorders. Despite immunosuppressive cytokines, such as transforming growth factor β and interleukin-10, which are found abundantly in the intestine induced by oral tolerance, the real mechanism by which it occurs remains controversial (Friedman A, Weiner H L. Induction of anergy or active suppression following oral tolerance is determined by antigen dosage. Proc Natl Acad Sci USA 1994;91:6688-92). Subcutaneous or intradermal injections of inhalant allergen extraction can provoke local or systemic reactions and was effective immunotherapy for patients with allergic rhinitis and asthma. Anaphylaxis develops occasionally and sometimes causes death (Passalacqua G, Albano M, Fregonese L, Riccio A, Pronato C, Canonica GW. Randomised controlled trial of local allergoid immunotherapy on allergic inflammation in mite-induced rhinoconjunctivitis. Lancet 1998;351:629-32). Oral or sublingual immunotherapy has been shown to be more effective and safer. The efficacy of sublingual-swallow route has been ascribed to the use of higher dose and partial “oral tolerance” mechanisms (Marth T, Strober W, Kelsall BL. High dose oral tolerance in ovalbumin TCR-transgenic mice. J Immunol 1996;157:2348-57; Gutgemann I, Fahrer A M, Altman J D, Davis M M, Chien Y H. Induction of rapid T cell activation and tolerance by systemic presentation of an orally administered antigen. Immunity 1998;8:667-73).

Induction of serum or mucosal antibody responses to orally administered antigens, however, may be problematic. Generally, such oral administration requires relatively large quantities of antigen since the amount of the antigen that is actually absorbed and capable of eliciting an immune response is usually low. Thus, the amount of antigen required for oral administration generally far exceeds that required for parenteral administration. Besides, purification of allergen is difficult and expensive; as a result, the application is restricted.

Recently, allergens produced by transgenic plant were enclosed by Mason et al., 1992 (Mason, H., D. Lam, and C. Amtzen. 1992. Expression of hepatitis B surface antigen in transgenic plants. PNAS. 89:11745-11749.). There are two types of expressing heterogenous proteins in plant, which are: (1) expressing heterogenous proteins in a transgenic plant that stably produces and accumulates proteins; and (2) expressing heterogenous proteins in a plant transfected by a virus that replicates, propagates, spreads and produces the desired proteins in the plant. Using. plant to produce allergens has many advantages in cost, safety and availability and is applied broadly in oral vaccines. In addition, oral vaccines produced in edible transgenic plants have other advantages of low cost and high safety and in that delivery, storage, and administration thereof are achieved in inexpensive and simple manner. Particularly, the selling price of the edible vaccine may be lowered to such a level that it can be easily purchased even in less developed countries.

Attempts to produce transgenic plants expressing bacterial and viral antigens have been made (Carrillo, C., A. Wigdorovitz, J. C. Oliveros, P. I. Zamorano, A. M. Sadir, N. Gomez, J. Salinas, J. M. Escribano, and M. V. Borca. 1998. Protective immune respones to foot-and-mouth disease virus with VP1 expressed in transgenic plants. J. Virol. 72:1688-1690. and Gilleland Jr, H. E., L. B. Gilleland, J. Staczek, R. N. Harty, A. Garcia-Sastre, P. Palese; F. R. Brennan, W. D. O. Hamilton, M. Bendahmane, and R. N. Beachy. 2000. Chimeric animal and plant viruses expressing epitopes of outer membrane protein F as a combined vaccine against

Pseudomonas aeruginosa lung infection. FEMS Immunology and Medical Microbiology. 27:291-297). However, until the work of the present inventors, no dust mite antigens, such as Der p 5 and Der p 2, had been expressed in plants. In particular, until the work of the present inventors, no such oral vaccine which were capable of eliciting an immune response as a mucosal immunogen had been obtained.

### SUMMARY OF THE INVENTION

The invention uses a transgenic plant to produce dust mite allergen.

Preferably, the dust mite allergen according to the invention can be an antigenic composition.

One subject of the invention is to provide a process for producing a dust mite allergen comprising the steps of:
(a) constructing a vector for plant transformation that comprises a DNA sequence encoding the dust mite allergen operably linked to a plant-specific promoter;
(b) transforming a plant cell or tissue with the vector of step (a); and
(c) obtaining the dust mite allergen from the plant cell or tissue of step (b).

In another aspect, the invention provides a process for producing an antigenic composition comprising a dust mite allergen, wherein the dust mite allergen is prepared by a process comprising the steps of:
(a) constructing a vector for plant transformation that comprises a DNA sequence encoding the dust mite allergen operably linked to a plant-specific promoter;
(b) transferring a plant cell or tissue with the vector of step (a); and
(c) obtaining the dust mite allergen from the plant cell or tissue of step (b).

Another object of the invention is to provide an antigenic composition comprising unpurified or partially purified recombinant dust mite allergen expressed in a plant at a level sufficient to induce an immunogenic response.

### BRIEF DESCRIPTION OF THE DRAWINGS

FIG. 1. Diagram of the nucleotide and amino acid sequence in the modified regions of viral genome of the viral vectors derived from a Taiwan strain (TW-TN3) of Zucchini yellow mosaic virus (ZYMV). Schematic representation of relevant portions of the genomic region of ZYMV non-coding regions (thick black lines), coding regions (open box), and the inserted foreign gene (black lines) are shown. Protease cleavage sites processed by the NIa protease of ZYMV are shown by “/”. The p35SZYMV2-26 that contained the full-length cDNA to the genomic ss(+)RNA of TW-TN3, driven by a Cauliflower mosaic virus (CaMV) 35S promoter to generate in vivo infectious transcript, was used for vector construction. An Nco I site was created between the N-terminal 2^{nd} and 3^{rd} aa of the HC-Pro coding sequence for insertion of foreign gene. In p35SZYMVGFPhis, the GFP coding sequence was inserted into the Nco I site and the NIa-protease cleavage site (S-V-R-L-Q/S) was inserted at the C-terminal end of the GFP to produce the free form GFP. In addition, several restriction enzyme sites and six histidines (His-tag) were engineered between the GFP and the NIa-protease cleavage site. In p35SZYMVDerp5, the coding sequence for the house dust mite allergen of Dermatophagoides pteronyssinus 5 (Der p 5) protein was inserted into the viral vector. The corresponding recombinant viruses generated by each construct are shown in parenthesis

FIG. 2 Western blot analysis of the virus-expressed vGFP and vDer p 5 and metal-affinity purification of His-tagged proteins from squash plants infected with ZYMV recombinants. A.) Extracts from equal amount (0.01 g) of leaf tissue collected at 7 dpi were loaded, separated on a gel (12.5%), and transferred onto nitrocellulose membrane. Simultaneously prepared blots were separately reacted with anti-GFP serum (A, lanes 1-5), anti-Der p 5 serum (A, lanes 6-8), or ZYMVV CP anti-serum. The recombinant viruses (described in FIG. 1) used for inoculation are shown above the membrane. B.) The affinity purification of His-tagged vGFP and vDer p 5 from plants infected with ZYMV-GFPhis (B, lanes 2-4) and ZYMV-Derp5 (B, lanes 6-8), respectively. Lanes 1 and 5, FT indicates the flow-through of the Ni²⁺-NTA column of the extract from infected plants; lanes 2 to 4, E1-3 indicates the consecutive 250 μL eluted fractions of vGFP, respectively. Lanes 6 to 8, E1-3 indicates the consecutive 250 μL eluted fractions of Der p 5, and M indicates protein markers. The vGFP/HC-Pro and vDer p 5/HC-Pro indicate the fusion form of VGFP and vDer p 5, respectively. The VGFP and vDer p 5 each/separately indicate the free form of vDer p 5. The CP indicates the ZYMV coat protein. The vGFP, vDer p 5, and ZYMV CP specific polyclonal antibody were used at a dilution of 1:4000, 1:4000, and 1:5000, respectively.

FIG. 3 Der p 5 specific IgG (white bars) and IgE (black bars) levels in serum of Der p 5-sensitized BALB/c mice challenged with inhalation vDer p 5 (0.1%) were determined with ELISA. Values are expressed as the mean±SEM. At least 12 mice were used in each experimental group. An asterisk(*) means p<0.05 as compared to vehicle-treated mice.

FIG. 4. Numbers of eosionphil, neutrophils, and mononulcear cells in the brochoalveolar lavage of Der p 5-sensitized mice treated with vehicle, low-dose of vDer p 5 (1 mg/kg/day for 10 days), high-dose of vDer p 5 (10 mg/kg/day for 10 days). Values are expressed as mean±SEM. Twelve mice were used for each experimental group. An asterisk(*) indicates p<0.05, as compared with vehicle-treated Der p 5-challenged mice.

FIG. 5. Der p 5 specific IgE levels in serum of Der p 5-sensitized BALB/c mice challenged with inhalation rDer p 5 were determined with

ELISA. * means p<0.1 as compared to vehicle-treated mice; ** means p<0.05 as compared to vehicle-treated mice; *** means p<0.001 as compared to vehicle-treated mice.

FIG. 6. Der p 5 specific IgG levels in serum of Der p 5-sensitized BALB/c mice challenged with inhalation rDer p 5 were determined with ELISA. * means p<0.1 as compared to vehicle-treated mice; ** means p<0.05 as compared to vehicle-treated mice; *** means p<0.001 as compared to vehicle-treated mice.

FIG. 7. Numbers of macrophages in the brochoalveolar lavage of Der p 5-sensitized mice. * means p<0.1 as compared to vehicle-treated mice; ** means p<0.05 as compared to vehicle-treated mice; *** means p<0.001 as compared to vehicle-treated mice.

FIG. 8. Numbers of neutrophils in the brochoalveolar lavage of Der p 5-sensitized mice. * means p<0.1 as compared to vehicle-treated mice; ** means p<0.05 as compared to vehicle-treated mice; *** means p<0.001 as compared to vehicle-treated mice.

FIG. 9. Numbers of eosinophils in the brochoalveolar lavage of Der p 5-sensitized mice. * means p<0.1 as compared to vehicle-treated mice; ** means p<0.05 as compared to vehicle-treated mice; *** means p<0.001 as compared to vehicle-treated mice.

FIG. 10. Comparison of Der p-5-specific IgE levels by feeding of ZYMV-Der p 5 with E. coli-Der p5 in allergen-sensitized BALB/c mice.

Mice were orally administered distilled water, ZYMV-Der p 5 and E. coli-Der p 5 per day for twenty-one days and challenged with 0.1% of Der p 5 at 21 days after sensitization. After 18 hours, serum was collected for determination of Der p-5-specific IgE; ¹Results are expressed as mean±SD for 6 mice in each group. *p<0.1 or **p<0.05 tested by Mann-Whitney U Test between ZYMV-Der p5 or E. coli-Der p5 versus control group, respectively.

FIG. 11. Enhancement of IFN-γ levels in BALF by feeding of ZYMV-Der p 5 or E. coli-Der p 5 in allergen-sensitized BALB/c mice. Mice were orally administered distilled water, ZYMV-Der p 5 and E. coli-Der p 5 per day for twenty-one days and challenged with 0.1% of Der p 5 at 21 days after sensitization. After 18 hours, BALF was collected for determination of IFN-γ levels; ¹Results are expressed as mean±SD for 6 mice in each group. *p<0.1 tested by Mann-Whitney U Test between ZYMV-Der p 5 or E. coli-Der p 5 versus control group, respectively.

### DETAILED DESCRIPTION OF THE INVENTION

In one aspect, the invention is to provide a process for producing dust mite allergen comprising the steps of:
(a) constructing a vector for plant transformation that comprises a DNA sequence encoding the dust mite allergen operably linked to a plant-specific promoter;
(b) transforming a plant cell or tissue with the vector of step (a); and
(c) obtaining the dust mite allergen from the plant cell or tissue of step (b).

The term “allergen” as used herein refers to an antigen that elicit hypersensitivity or allergic reactions. According to the invention, dust mite allergens comprise but are not limited in Dermatophogoides farinae (known as Der f) and Dermatophagoides pteronyssinus (known as Der p) allergens, or the mixture thereof; and wherein preferably, the allergens comprise Der p 5 and Der p 2 allergens. In order to raise the expression amount of the allergen, the allergen can further comprise an endoplasmic reticulum (ER) retention signal peptide which leads to accumulation of the recombinant allergen on ER.

The term “allergy” as used herein refers to the systematic reaction to a normal innocuous environmental antigen. It results from the interaction between the antigen and antibody or T cells produced by earlier exposure to the same antigen. The term “allergic reaction” as used herein refers to a response to innocucous environmental antigens or allergens due to pre-existing antibody or T cells. There are various immune mechanisms of allergic reactions, but the most common one type is the binding of allergen to IgE antibody on mast cells that causes asthma, hay fever, and other common allergic reactions.

According to the invention, the vector for plant transformation comprises a DNA sequence encoding the dust mite allergen operably linked to a plant-specific promoter.

In one embodiment of the invention, the vector for plant transformation is based on a conventional vector in plant, e.g., an ordinary binary vector, a cointegration vector or a vector designed to express in plant without T-DNA region.

In another embodiment of the invention, the vector for plant transformation is a modified plant virus. Potyviruses are usually utilized for the purpose, and preferably, zucchini yellow mosaic virus (ZYMV) and tobacco mosaic virus (TMV) are suitable according to the invention. In order to transform plants, the allergen gene must be inserted into the genome of the plant. Furthermore, the allergen gene must contain all the genetic control sequences necessary for the expression of the gene after it has been incorporated into the plant genome. Accordingly, a vector must be constructed to provide the regulatory sequences such that they will be functional upon inserting a desired gene. In one embodiment of the invention, the regulatory sequences comprise an operably linked plant expressible promoter, a translation initiation codon (ATG) and a plant functional poly(A) addition signal (AATAAA) 3′ of its translation termination coding. Additionally, in order to obtain a higher level of expression, untranslated regions 5′ and 3′ to the inserted genes are provided. When the expression vector/insert construct is assembled, it is used to transform plant cells which are parts of a mature plant or have an ability to regenerate a new plant. These transgenic plants carry the viral gene in the expression vector/insert construct. Once the virus replicates, propagates and spreads, the allergens are produced in the plant.

The term “operably linked” refers to the linking of nucleotide regions encoding specific genetic information such that the nucleotide regions are contiguous, and the functionality of the region is preserved and will perform its function relative to the other regions as part of a functional unit.

Promoters which are known or found to cause transcription of a foreign gene in plant cells can be used in the present invention. Such promoters may be obtained from plants or viruses, and for example, the 35S. promoter of cauliflower mosaic virus (CaMV) (as used herein, the expression “CaMV 35S” promoter includes variations of CaMV 35S promoter, e.g., promoters derived by means of ligations with operator regions, random or controlled mutagenesis, etc.). Furthermore, the promoters according to the invention can regulate high expression in edible plant parts.

In a preferred embodiment of the invention, the vector comprises a gene for a selectable marker gene such as an antibiotic-resistance gene (e.g., a kanamycin-resistance gene), a herbicide-resistance gene, a metabolic pathway-related gene, a gene relating to the physical properties, a gene encoding a luciferase (such as GFP), a gene encoding a β-glucuronidase (GUS) or a gene encoding a β-galactosidase, etc. Once the host plant has been selected and the method of gene transfer into the plant has been determined, a constitutive, a developmentally regulated, or a tissue specific promoter for the host plant is selected so that the allergen is expressed in the desired part(s) of the plant.

Preferably, the vector is ZYMV. According to the invention, the ZYMV-Der p 5 that expressed the free form of Der p 5 is highly stable over one-year observation after 20 transfers, and no deletion variants were noticed. Therefore, the ZYMV-base viral vector permits both systemic spread and efficient, stable expression of foreign proteins. It is considered that the stability may be greatly dependent upon the nucleotide sequence and the length of the insert (Gal-On A, Canto T, Palukaitis P. Characterization of genetically modified Cucumber mosaic virus expressing histidine-tagged 1a and 2a proteins. Arch Virol 2000;145(1):37-50).

According to the invention, plants utilized include any dicotyledonous plant and monocotyledonous plant. In a preferred embodiment, a part or whole plant according to the invention is edible, which plants include, but are not limited in tobacco, potato, zucchini squash, tomato, lettuce, white grape, banana, rice, radish, carrot, apple, soybean, corn, and berries. More preferably, the plants according to the invention include Kennebec variety of potato, Nicotina benthamiana and Cucurbia pepo L. var. Zucchini.

The choice of the plant cell or tissue for transformation depends on the nature of the host plant and the method for transformation. In one embodiment of the invention, the tissue is regenerable, which retains the ability to regenerate whole, fertile plants following transformation. For example, the plant tissue includes callus, suspension culture cells, protoplasts, leaf segments, stem segments, tassels, pollen, embryos, hypocotyls, tuber segments, meristematic regions, and the like. In another embodiment of the invention, the tissue is part of a mature plant. Preferably, the tissue is edible or has an ability to express and/or purifying enormously the allergens according to the invention. For example, the tissue includes leaves, fruits, stems, tubers, and the like.

According to the invention, the step of transforming the plant cell or tissue with the vector includes (1) Agrobacterium-mediated gene transferring; (2) direct DNA uptaking; or (3) plant virus infecting.

The Agrobacterium system is especially viable in the creation of transgenic dicotyledenous plants. In the preferred embodiment of the present invention, the Agrobacterium-Ti plasmid system is utilized. The tumor-inducing (Ti) plasmids of A. tumefaciens contain a transforming DNA (T-DNA) which is transferred to plant cells and then integrates into the plant host genome with the help of inducible virulence (vir) genes of Agrobacterium. The vector comprising the allergen gene, T-DNA region and a selectable marker gene can be constructed in Escherichia coli and then transferred into Agrobacterium via a conjugation mating or direct uptaking by Agrobacterium. Those skilled in the art should recognize that there are many Agrobacterium strains, such as A. tumefaciens and A. rhizogenes, and plasmid constructions that can be used to optimize genetic transformation of plants. According to the invention, those skilled in the art can choose the method for inoculation depending upon the plant species and the Aarobacterium delivery system; for example, leaf disc procedure or in vitro transformation of regenerating protoplasts.

According to the invention, direct physical method of introducing foreign DNA into the plant cells can also applied. In electroporation, the protoplasts are briefly exposed to a strong electric field. In microinjection, the DNA is mechanically injected directly into the cells using micropipettes. In microparticle bombardment, the DNA is adsorbed on microprojectiles such as magnesium sulfate crystals or tungsten particles. Direct incubation of DNA with germinating pollen is also included.

According to the invention, when using the modified plant viruses as vectors, the viruses can be utilized to infect plants at wound sites.

Optionally, the process according to the invention further comprises a step of regenerating a transgenic plant from the plant cell or tissue before step (c). The plant cell or tissue transformed is then regenerated to form a transgenic plant. As used herein, the term “regeneration” refers to growing a whole plant from a plant cell, a group of plant cells or a plant part. The methods for plant regeneration are well known to those skilled in the art. When transformation is of an organ part, regeneration can be from the plant callus, explants, organs or parts. Such methods for regeneration are also known to those skilled in the art.

There are several strategies for obtaining the dust mite allergen from plant cells or whole plants. In one embodiment, the method of obtaining the allergen according to the invention is accomplished by obtaining the plant cell or whole plant or portions thereof such as fruits, leaves, stems, and tubers or extract thereof. In another embodiment, the dust mite allergen is provided by further purifying the allergen from the extract. In still another embodiment, the dust mite allergen is obtained by merely harvesting at least a part of a transgenic plant, such as fruit or seeds. In still another embodiment, the dust mite allergen is provided in the form of the transgenic plant itself.

Another object of the invention is to provide a process for producing an antigenic composition comprising a dust mite allergen, wherein the dust mite allergen is prepared by a process comprising the steps of:
(a) constructing a vector for plant transformation that comprises a DNA sequence encoding the dust mite allergen operably linked to a plant-specific promoter;
(b) transforming a plant cell or tissue with the vector of step (a); and
(c) obtaining the dust mite allergen from the plant cell or tissue of step (b).

Also claimed in the invention is an antigenic composition comprising unpurified or partial purified dust mite allergen expressed in a plant at a level sufficient to induce an immunogenic response.

In the animal model, the antigenic composition according to the invention has great effect on treating mice sensitized with dust mite allergens in the histological examining the lung tissue of the mice. The amount of dust mite specific IgE was lower, which shows that the allergic reaction was regulated. Besides, lung function of the mice after oral administrating the antigenic composition was also recovered.

The present invention overcomes the deficiencies of the prior art in producing the antigenic composition in one or more tissues of a transgenic plant (such as edible fruit, juice, grains, leaves, tubers, stems, seeds, roots or other plant parts). The present invention provides an inexpensive means for production and administration of antigenic composition. Expenses for purification and adverse reactions are thereby avoided. In addition, the antigenic products produced from edible transgenic plants have other advantages; for example, the delivery, storage, and administration are achieved in inexpensive, simple and safe manners. Furthermore, the effect of inhibition of dust mite specific IgE levels of the antigenic composition according to the invention is better than that of the conventional composition such as an antigenic composition comprising a dust mite allergen produced by Eschenchia coli.

The following Examples are given for the purpose of illustration only and are not intended to limit the scope of the present invention.

### EXAMPLE 1

### Expression Dust Mite Allergen in A Transgenic Plant

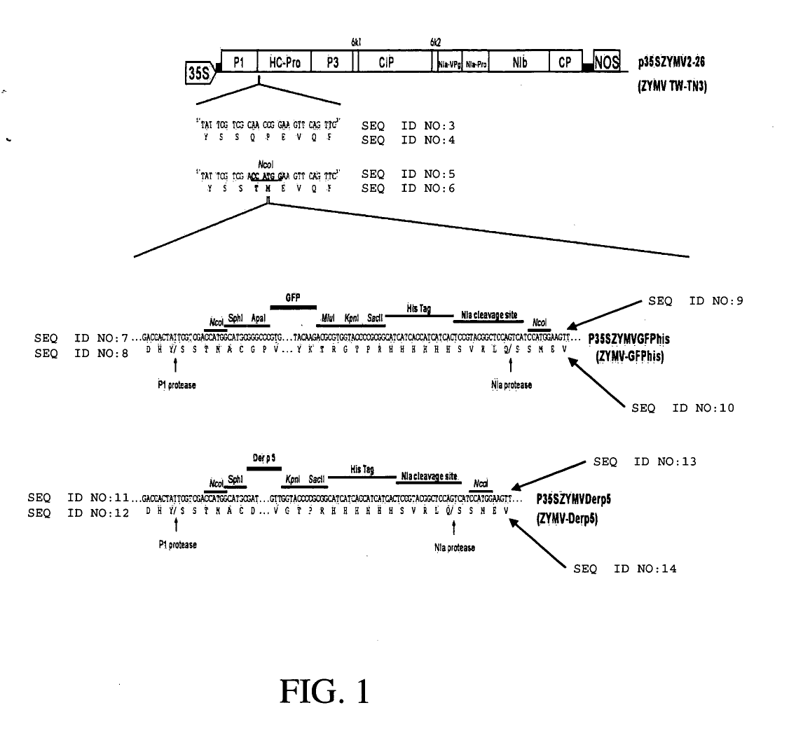

Generation of ZYMV-Der p 5 recombinant plant virus: The development of ZYMV vector was based on the previously constructed infectious clone, p35SZYMV2-26 (Lin S S, Hou R F, Yeh S D. Construction of in vitro and in vivo infectious transcripts of a Taiwan strain of Zucchini yellow mosaic virus. Bot Bull Acad Sin 2002;43:261-269), which is driven by a Cauliflower mosaic virus (CaMV) 35S promoter to generate in vivo infectious transcript, to insert the ORF of GFP (Clontech) between the P1 and HC-Pro coding regions of ZYMV. The multiple cloning sites (Nco I, Sph I, Apa I, Mlu I, Kpn I, and Sac II) were created flanking the N- and C-terminis of GFP coding region by polymerase chain reaction (PCR) with designed primers. A hexahitidine (histidine-tag) and NIa protease motif of TW-TN3 (S-V-R-L-Q/S) were also created by PCR on the C-terminal end of GFP ORF. The new viral vector, harboring the report gene GFP, multiple cloning sites, a histidine-tag, and a NIa protease cleavage motif, was designated as p35ZYMVGFPhis (FIG. 1).

The Der p 5 cDNA was amplified using reverse transcriptase-polymerase chain reaction (RT-PCR) from total RNA of mite crude extraction and the Sph I and Kpn I sites were created flanking the 5′- and 3′ end of Der p 5 ORF, respectively. The RT-PCR product was digested with Sph I and Kpn I and then ligated with Shp I-Kpn I digested p35SZYMVGFPhis to generate p35ZYMVDerp5 (FIG. 1).

Plant inoculation: The systemic host plants of Cucurbita pepo L. var. Zucchini at two cotyledons stage and local lesion host plants of Chenopodium quinoa Willd. with four fully expanded leaves were used for infectivity assay of the various constructs. Individual plasmids (1 μg) containing recombinant infectious clones were used to infect C. quinoa plants by mechanical rubbing on leaves (Lin S S, Hou R F, Yeh S D. Construction of in vitro and in vivo infectious transcripts of a Taiwan strain of Zucchini yellow mosaic virus. Bot Bull Acad Sin 2002;43:261-269). Seven days post inoculation (dpi), single lesions were isolated and mechanically transferred to plants of the systemic host zucchini squash. Inoculated plants were kept in a temperature-controlled greenhouse (23-28° C.) for observation.

Western blot analyses: The GFP protein also was expressed by pET36b (Novagen) (bacterial expressed GFP, bGFP) and purified by gel-purification. The Der p 5 protein was expressed by pGEX-2T (Promega) (bacterial expressed Der p 5, bDer p 5) and was purified by GST-affinity column (Hsu C H, Chua K Y, Huang S K, Hsieh K H. Immunoprophylaxis of allergen-induced IgE synthesis and airway hyperresponsiveness in vivo by genetic immunization. Nature Med 1996;2:540-4). The production of polyclonal antisera to E. coli expressed bGFP and bDer p 5 following the method described previously (Lin S S, Hou R F, Yeh S D. Construction of in vitro and. in vivo infectious transcripts of a Taiwan strain of Zucchini yellow mosaic virus. Bot Bull Acad Sin 2002;43:261-269). Antisera to ZYMV CP, bGFP, and bDer p 5 for immunoblot analyses were used at a 1:5000 dilution. vGFP and vDer p. 5 protein concentrations in plant extracts were determined by the Image Gange version 2.54 software (Fuji Photo Filn co., Minat-Ku, Tokyo, Japan) using the BSA protein as standard.

Isolation of the histidine-tagged proteins from the infected plants: Histidine-tagged vGFP and vDer p 5 proteins expressed by the constructed ZYMV viral vectors p35SZYMVGFPhis- or p35SZYMVDerp5-, respectively, and were purified by affinity column. Leaves (20 g) of the recombinant infected squash were harvested 8-10 dpi, and the target proteins were isolated with Ni²⁺-NTA agarose (Qiagen Inc., Stanford, Valencia, Calif.) by the method described (Hsu C H, Chua K Y, Huang S K, Hsieh K H. Imrnunoprophylaxis of allergen-induced IgE synthesis and airway hyperresponsiveness in vivo by genetic immunization. Nature Med 1996;2:540-4). The purified proteins were analyzed by SDS polyacrylamide gel (12.5%) electrophoresis and stained with Coomassie brilliant bule R-250 (Sigma) or subjected to inmunoblot analysis.

Concentration of vDer p 5 protein in squash extracts for animal test: Leaves of squash plants (1 kg) infected with ZYMV-Derp5 recombinant virus were harvested 10 dpi and homogenized with a blender, each with 250 g tissue, in twice the sample volumes of water. The homogenate was clarified by centrifugation at 5,000 g for 10 rnin, filtered through Miracloth (Calbiochem, La Jolla, Calif.), and centrifuged again at 40,000 g for 30 min. The concentration of extracted vDer p 5 was determined by ELISA using the antiserum to bDer p 5. The supernatants were lyophilized, and the total protein concentrates were distributed into vials for animal testing.

Der p 5 protein determination: A 96-well plate (Nunc®) was coated with 100 ml of serial dilution of Der p 5 (dry weight concentration origin at 20 mg/ml and final at 156.25 mg/ml) in 0.1 M sodium phosphate buffer (pH 9.6).at 37° C. for 2 h. The plate was washed 3 times with PBS contained 0.05% Tween 20 and blocked with 1% BSA in PBS at 37° C. for 2 h. 100 ml of diluted rabbit anti-Der p5 IgG 1:2000 in PBS contained 1% BSA was added and incubated at 37° C. for 2 h. After washing, the plate was incubated with goat anti-rabbit IgG-conjugated alkaline phosphatase (1:2000, ZyMax® 81-6122) at 37° C. for 1 h. The plate was washed and incubated for 30 min with pNPP (Sigma®) substrate and the color reaction was measured at 405 nm. Recombinant Der p5-6x-his protein concentration was determined by BIO-RAD® protein assay and Der p5 concentration calibration curve could be determined.

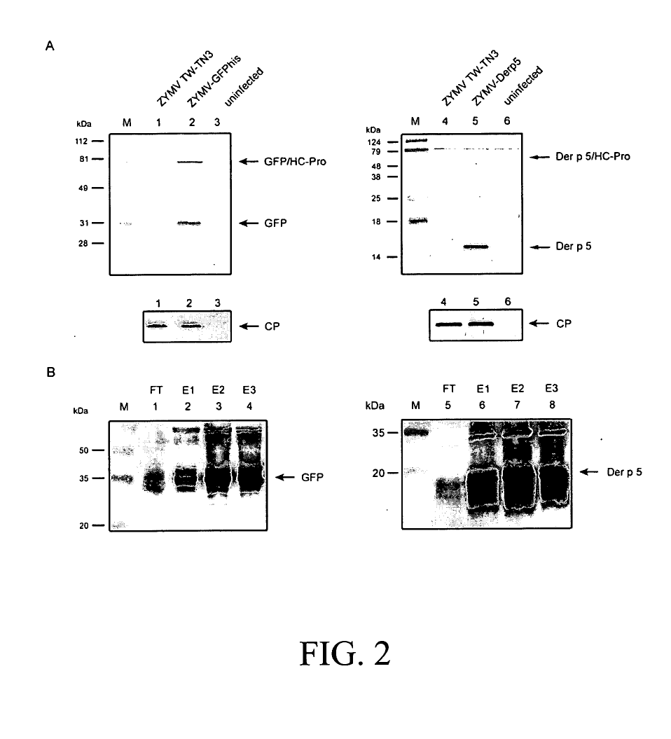

GFP with histidine-tag was readily purified by the Ni-NTA column and recognized by GFP-specific polyclonal antibodies in imnunoblot analysis (FIG. 2B, lanes 2, 3 and 4). Purified GFP protein absorbed by the Ni-NTA column from squash extracts at 10 dpi was estimated about 3.7 μg per gram of the leaf tissue by Image Gange software. Der p 5 protein was also purified by the Ni-NTA column from 20 g leaves of infected squash and readily recognized by Der p 5-specific polyclonal antibodies in imnmunoblot analysis (FIG. 2B, lanes 6, 7 and 8). Purified Der p 5 protein from infected squash at 10 dpi was estimated as 1.5 μg per gram of the leaf tissue.

### EXAMPLE 2

### Animal Model of Treating Purified Der p 5

Animals and Study Protocol: Female BALB/c mice, aged between 6 and 8 weeks, obtained from the animal-breeding center of the College of Medicine, National Taiwan University (originated from The Jackson Laboratory, Bar Harbor, Me.), were divided into four groups for each experiment (Table 1). Mice were actively sensitized by intraperitoneal injection of 10 μg. of bDer p 5 with 4 mg of aluminium hydroxide (Wyeth Pharmaceuticals, Punchbowl, Australia). 14 and 21 days after the initial sensitization, mice were exposed to an aerosol of 0.1% of bDer p 5 purified from E. coli for 20 min. Aerosols were generated with an ultrasonic nebulizer (Devilbiss, Somerset, Pa.). The mean mass diameter of the aerosol was less than 4 μm. Eight hours after last inhalation challenge, the bronchoalveolar lavage fluids (BALF) and sera were collected. Seven days after sensitization, mice were treated with vDer p 5 (low-dose group, 1 mg/kg/Day; high-dose group, 10 mg/kg/day) concentrated from ZYMV-infected squash for 10 days orally for 10 days. The concentration of vDer p 5 in crude extraction of ZYMV-infected squash equals to 25 μg/gm. Control groups were treated with PBS only.

TABLE 1
Characteristics of 4 experimental groups
and procedures performed for each group

| | | Body | | | |
|---|---|---|---|---|---|
| | | Weight | | | Aerosol |
| Group | No. | (gm) | Sensitization | Treatment | challenge |
| C | 12 | 28 ± 1.1 | bDer p 5 | PBS | NT |
| NC | 12 | 30 ± 1.5 | bDer p 5 | PBS | bDer p 5 |
| Low- | 18 | 29 ± 1.2 | bDer p 5 | vDer p 5 | bDer p 5 |
| dose | | | | (1 mg/kg/day) | |
| High- | 18 | 32 ± 1.4 | bDer p 5 | vDer p 5 | bDer p 5 |
| Dose | | | | (10 mg/kg/day) | |

Determination of Der p 5-specific IgG2a and IgE: The amounts of Der p 5-specific IgG2a, and IgE were determined by ELISA. Protein high-binding plates were coated with 100 μl of purified bDer p 5 or vDer p 5 diluted in coating buffer (0.1 M NaHCO₃, pH 8.2) at a concentration of 5 μg/ml. After overnight incubation at 4° C., plates were washed 3 times and blocked with 3% (wt/vol) BSA-PBS buffer for 2 h at 25° C. Sera were used at 1:100 dilution for IgG measurement and 1:10 dilution for IgE measurement in duplicate. After overnight incubation at 4° C., biotinylated rat anti-mouse IgE monoclonal antibody (R35-72, PharMingen), or rat anti-mouse IgG mAb (R12-4, PharMingen) diluted in 0.05% gelatin buffer, was added and incubated for an additional hour. Avidin-alkaline phosphatase conjugate was then added (1:1000) and incubated for 1 h at 25° C. After 6 washes, color reaction was imitiated with the addition of phosphatase substrate p-nitrophenyl phosphate (1 mg/ml) disodium salt (Sigrna). Plates were read in a microplate autoreader (Metertech, Taiwan) at 405 nm. Readings were referenced to a standard serum pooled from 6 mice which were initially i.p. injected with 10 μg of bDer p 5 with aluminum hydroxide and boosted after 21 d with the same dose. The standard senum was calculated as 100 ELISA units/ml.

Bronchoalveolar larvage and cell counting After measurement of lung-function parameters, mice were lavaged with 5×0.5-ml aliquots of 0.9% sterile saline through a polyethylene tube introduced through a tracheostomy. Lavage fluid was centrifuged (500 g for 10 min at 4° C.), and the cell pellet was resuspended in 0.5 ml of Hank's balanced salt solution. Total cell counts were made by adding 10 [l of the cell suspension to 90 μl of 0.4% trypan blue, and counted under a light microscope in a Neubauer chamber. Differentiated cell counts were made from cytospin preparations stained by Leu's stain. Cells were identified and differentiated into eosinophils, lymphocytes, neutrophils, and macrophages by standard morphologic techniques, and 500 cells were counted under 400-fold magnification and the percentage and absolute number of each cell type were determined.

Statistical analvsis: To assay the changes of IgE and IgG levels, and cells in the BALF after bDer p 5 challenges, repeated measures for analysis of variance (ANOVA) were performed to compare the differences between the groups. After analysis of variance, Duncan multiple range tests was used to differentiate differences between experimental and control groups. A value of p<0.05 was used to indicate a statistically significant difference.

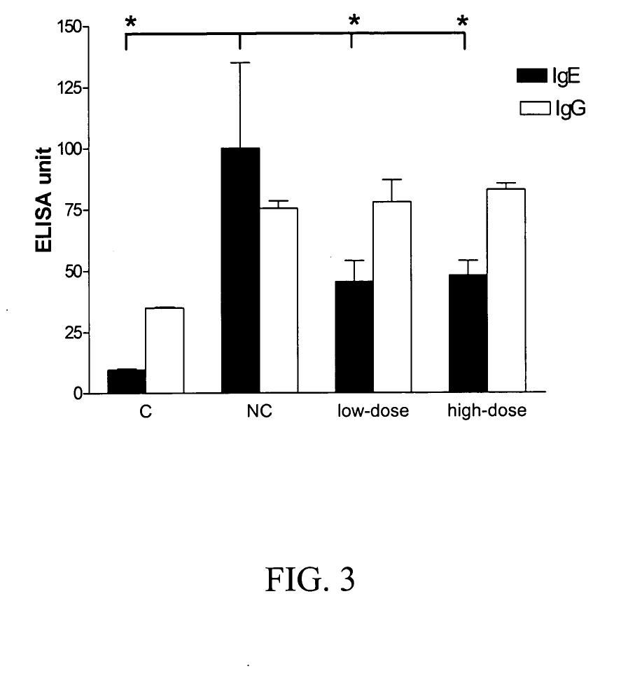

The in vivo efficacy of oral administration of recombinant Der p 5 was evaluated to determine whether a protective response to inhalational allergen challenge was functionally significant. Both mock-treated and rDer p 5-treated mice received two inhalational challenges with allergen Der p 5 two and three weeks after intraperitoneally sensitization. The presence of anti-Der p 5 IgE in the serum three weeks after allergen challenge was assayed by an ELISA. Der p 5-specific IgE increased significantly in the mock-treated group; in contrast, rDer p 5-treated mice showed more than 50% inhibition of Der p 5-specific IgE synthesis (FIG. 3). The inhibition of IgE production in the mice orally fed with rDer p 5 was specific to Der p 5, because rDer p 2-challenged mice could produce Der p 2-specific IgE. Thus, an oral administration of rDer p 5 expressed by ZYMV in squash could inhibit an in vivo allergen-specific IgE production efficiently and in an allergen-specific manner. There was no significant difference in specific IgG levels between the groups (FIG. 3).

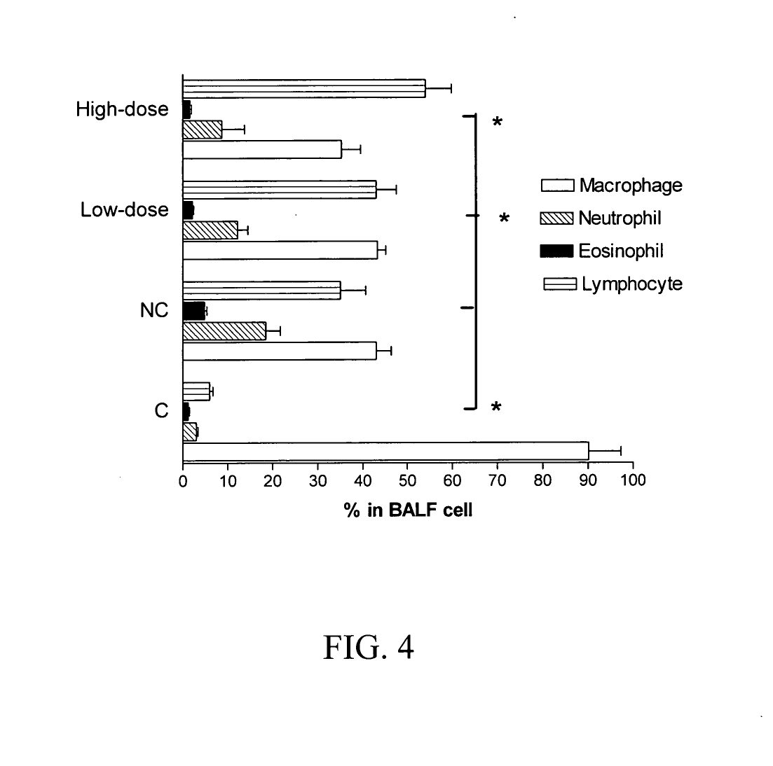

To investigate whether an oral administration of rDer p 5 can suppress allergen-specific airway inflammation, the number of cells in the brochoalveolar lavage (BALF) was used as a measure for the infiltration of cells in the airways (FIG. 4). A significantly low number of eosinophils and neutrophils in the BALF of rDer p 5-treated mice were observed, when compared to mock-treated groups (p<0.05). The numbers of macrophage and lymphocyte were not different between the groups. Therefore, Der p 5 inhalational challenge induced an eosinophilic and neutophilic cellular infiltrate in the BALF. This inflammation could be inhibited by an oral administration of rDer p 5, but not by PBS only.

### EXAMPLE 3

### Animal Model of Treating Leaves from Transgenic Plant Expressing Der p 5

Animals and Study Protocol: Female mice BALB/c, aged between 6 and 8 weeks, were obtained from the animal-breeding center of the College of Medicine, National Taiwan University (originated from The Jackson Laboratory, Bar Harbor, Me.), and were divided into 6 groups for the experiments shown in Table 2; wherein C represented Normal Control; NC represented Negative Control, in which the mice were sensitized and fed with ZYMV leaves (2 g/kg); PC represented Positive Control, in which the mice were sensitized and fed with eN-Lac (Lactobacillus paracasei, which was proved to effect on treating allergy) 10¹²/day; Low-dose presents that the mice were sensitized and fed with ZYMV-Der p 5 leaves (200 mg/kg/day); High-dose presents that the mice were sensitized and fed with ZYMV-Der p 5 leaves (2 g/kg/day). Animals were actively sensitized by intraperitoneal injection of 10 μg of Der p 5 purified from E. coli with 4 mg of aluminium hydroxide (Wyeth Pharmaceuticals®, Punchbowl, Australia). After the sensitization, animals were fed with leaves of ZYMV, ZYMV-Dp5 obtained in Example 1 or eN-Lac once a day for 4 weeks.

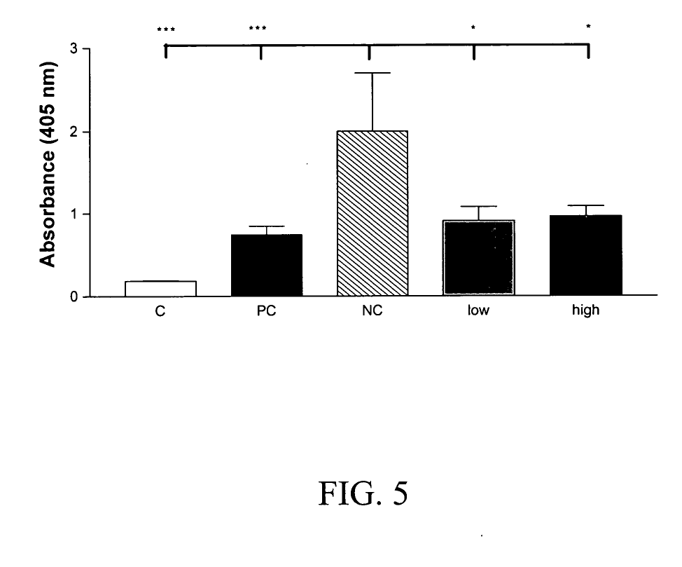

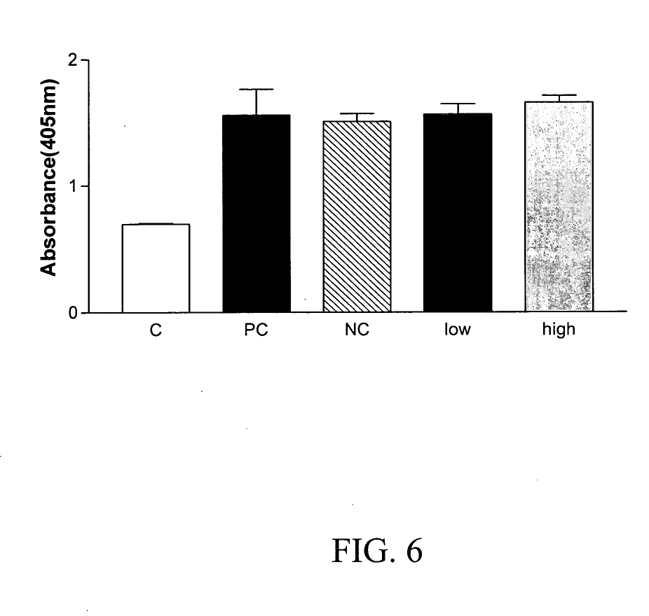

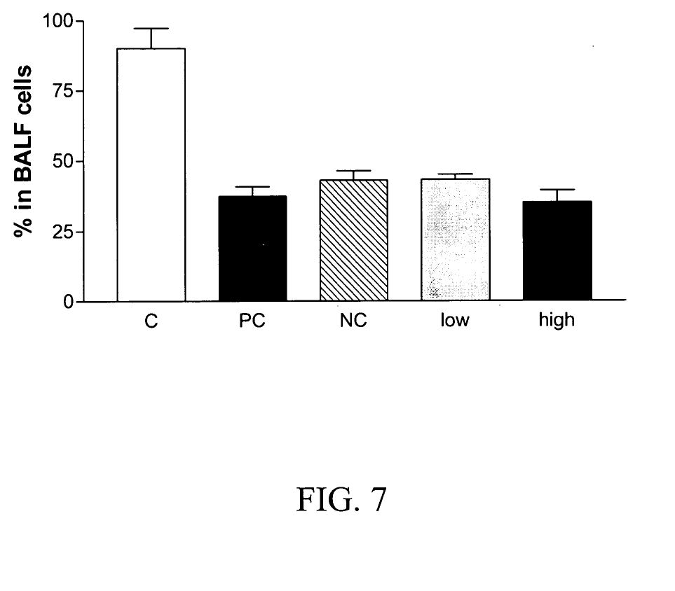

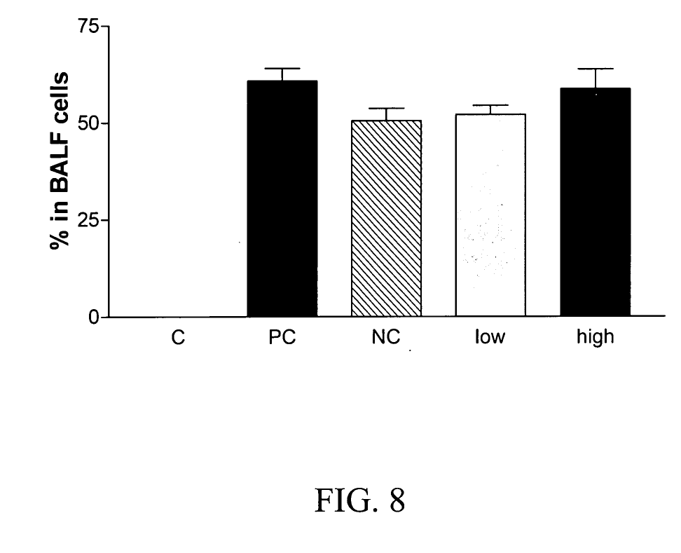

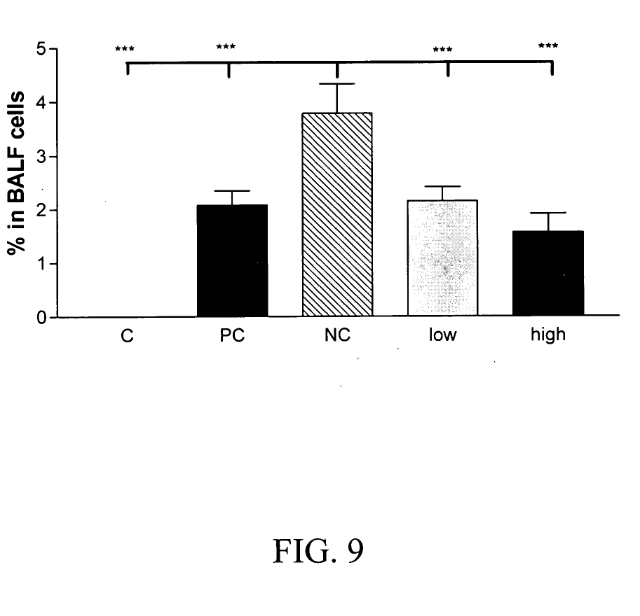

Determination of Der p 5-speciflic IgG2a and IgE and bronchoalveolar larvage and cell counting: The amounts of Der p 5-specific IgG2, IgE and bronchoalveolar larvage cell counting were determined by ELISA as described in Example 2 and shown in Table 2 and FIGS. 5 to 9. The results were subjected to Kruskal-Wallis H Test which used Dunnet Test and N. C as baseline.

TABLE 2

| | | | | Low- | High- | |
|---|---|---|---|---|---|---|
| | N. C | Control | P. C | dose | dose | P Value |
| IgE | 2.00 ± 0.70 | 0.19 ± 0.01 | 0.74 ± 0.11 | 0.91 ± 0.17 | 0.96 ± 0.12 | 0.026**^{a,b,c,d} |
| IgG | 1.51 ± 0.06 | 0.70 ± 0.006 | 1.73 ± 0.06 | 1.56 ± 0.18 | 1.66 ± 0.05 | 0.000***^{a,b} |
| Marcophage | 43.01 ± 3.35 | 90.10 ± 7.20 | 37.45 ± 3.38 | 43.32 ± 1.84 | 35.19 ± 4.30 | 0.000***^{a} |
| Lymphocyte | 6.11 ± 0.99 | 9.85 ± 7.15 | 8.59 ± 1.10 | 4.88 ± 0.84 | 6.90 ± 1.37 | 0.137 |
| Neutrophil | 50.55 ± 3.19 | 0.00 ± 0.00 | 60.76 ± 3.20 | 52.18 ± 2.31 | 58.72 ± 5.06 | 0.000***^{a,b} |
| Eosinophil | 3.79 ± 0.55 | 0.00 ± 0.00 | 2.08 ± 0.27 | 2.11 ± 0.29 | 1.57 ± 0.35 | 0.000***^{a,b,c} |

^{a},^{b},^{c}, and ^{d} showed Significance difference in Control, P. C, low-dose, and high-dose groups, respectively.
*P < 0.1,
**P < 0.05,
***P < 0.001

The results showed that the amounts of Der p 5 specific IgE of the groups treated with the ZYMV-Dp5 leaves were significantly lower than those of the control group and dose-dependent. In contract, the amounts of the Der p 5 specific IgG of the groups treated with the ZYMV-Dp5 leaves were raised. It demonstrated that ZYMV-Dp5 leaves could inhibit the production IgE antibodies associated with allergy.

The results also showed that the numbers of eosinophils of the groups treated with the ZYMV-Dp5 leaves decreased. In contract, the numbers of T cells and monocytes of the group treated with the ZYMV-Dp5 leaves increased significantly and were dose-dependent.

### EXAMPLE 4

### Comparison of IgE-inhibiting Activitv of ZYMV-Der p5 with E. coli-Der p5 in Allergen-Induced a Asthmatic Murine Model

Animals and Stud Protocol: Female 7 week-old BALB/c mice were purchased from National Laboratory Animal Center (Taipei, Taiwan). All animals were maintained individually in cages with a controlled temperature (24±2° C.) and a humidity (60±5%) and maintained on a 12 h light-dark cycle under specific-pathogen-free conditions. Five groups were performed in this study: Group 1, normal mice; Group 2, control group feed with Ig ZYMV /Kg B.W. once a day; Group 3 feed with 1 g ZYMV-Der p 5/Kg B.W. once a day; Group 4, control group; and Group 5 feed with 12.83 mg E. coli-Der p5/Kg B.W. once a day. Animals were allowed free access to diets and water. ZYMV, ZYMV-Der p 5 and E. coli-Der p 5 were supplied by oral tube for twenty-one days after sensitization. The groups of the animals except group 1 were sensitized by i.p. injection of 10 μg recombinant Dermatophagoides pteronyssinus allergen Der p 5-6 x his-tag fusion protein with 4 mg of aluminum hydroxide. Fourteen days after sensitization, mice were boosted with the same dosage as sensitization. On the twenty-first day after sensitization, an inhalation challenge was perf6rmed. Briefly, animals except group1 were exposed to an aerosol of 0.1% of Der p 5-6× his-tag fusion protein diluted in PBS. After 18 hours, serum was collected by tail vein bleeding of each mouse, and the levels of IgE, IgG1 and IgG2a were determined by ELISA.

Determination of serum specific antibody and IFN-γ levels in BALF by ELISA: The levels of Der p 5-specific IgG1, IgG2a and IgE in serum or IFN-γ in BALF were determined by ELISA. A 96-well plate (NUNC) was coated with 150 μl of Der p 5 (10 μg/ml) in sodium carbonate buffer (pH 9.6) or anti-mouse IFN-γ (2 μg/ml, Pharmingen, USA) in 0.1 M sodium phosphate buffer (pH 9) at 4° C. overnight. Following the coating step, PBS containing 3% BSA was used to block nonspecific binding and incubated for 2 h. at room temperature (RT). The plate was washed with PBS containing 0.05% Tween 20. 100 μl of diluted test serum (1:10 dilution in PBS containing 1% BSA for IgG1, IgG2a and 1:5 dilution for IgE) or 150 μl of BALF was added to each well and incubated for 2 h. at RT. The plate was washed and incubated with biotin-conjugated anti-mouse IgG1, IgG2a, IgE (1:2,000) and IFN-γ (0.25 μg/ml, Pharmingen, USA) for 2 h. at RT. After washing the plate, 200 μl of streptavidin-conjugated alkaline phosphatase (1:2,000) was added to each well and incubated for 1 h. at RT. The pNPP substrate (p-Nitrophenylphosphate, disodium, Sigma, USA) was added and the value of optical density was detected at 405 nm for each sample.

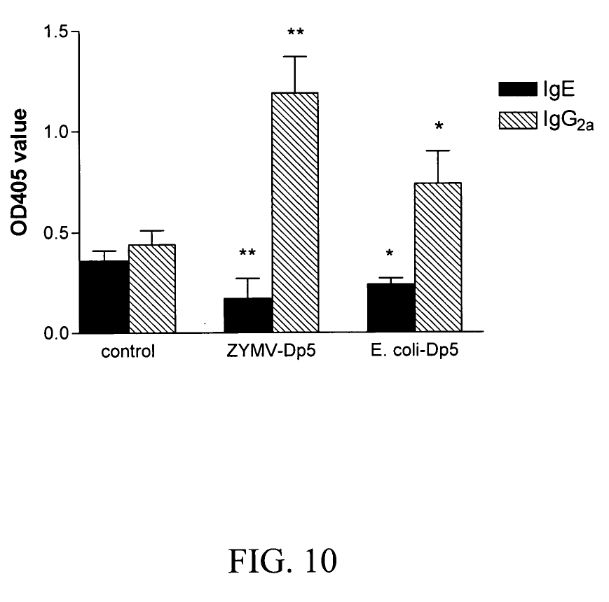

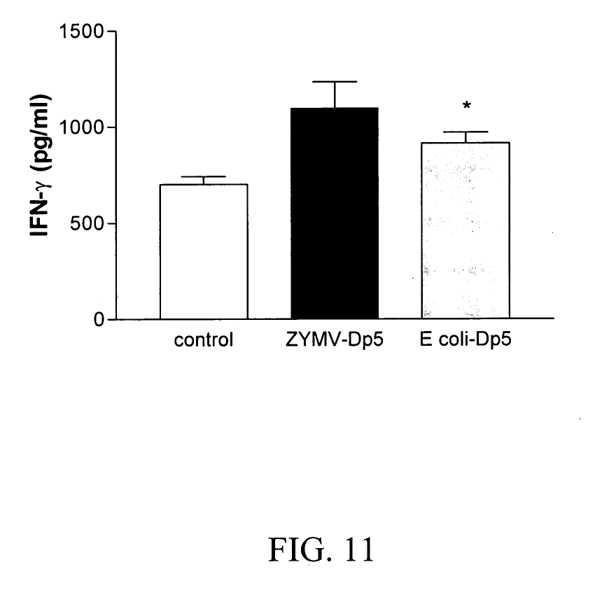

Result: The presence of anti-Der p 5 IgE in the serum after inhalation challenge was tested by ELISA. Der p 5-specific IgE increased significantly in the ZYMV-treated group compared to normal group (p<0.05); in contrast, ZYMV-Der p5 treated mice showed a significant inhibition of Der p 5-specific IgE synthesis (p<0.05) (Table 2). E. coli-Der p 5 (in which the dosage is the same as that of ZYMV-Der p 5) also showed a significant inhibition in Der p 5-specific IgE (p<0.05), but ZYMV-Der p 5 showed an improved effect in inhibition of Der p 5-specific IgE production as compared to E. coli-Der p 5 (p<0.1). Also, ZYMV-Der p 5-treated mice showed a significant increase in Thl-type Der p 5-specific IgG2a than E. coli-Der p 5-treated mice (p<0.1) (FIG. 10). Thus, feeding of ZYMV-Der p 5 could inhibit allergen-specific IgE production more efficiently than E. coli-Der p 5. The concentration of IFN-γ in bronchoalveolar lavage fluids (BALF) was determined after inhalation challenge. IFN-65 production increased in ZYMV-Der p 5, significantly in E. coli-Der p 5 (p<0.1), as compared to control group (group 2) (FIG. 11).

While embodiments of the present invention have been illustrated and described, various modifications and improvements can be made by persons skilled in the art. It is intended that the present invention is not limited to the particular forms as illustrated, and that all the modifications not departing from the spirit and scope of the present invention are within the scope as defined in the appended claims.

1. A process for producing a dust mite allergen comprising the steps of:
(a) constructing a vector for plant transformation that comprises a DNA sequence encoding the dust mite allergen operably linked to a plant-specific promoter;
(b) transforming a plant cell or tissue with the vector of step (a); and
(c) obtaining the dust mite allergen from the plant cell or tissue of step (b).

2. The process of claim 1, wherein the dust mite allergen is selected from the group consisting of Der p 5 and Der p 2 allergens.

3. The process of claim 1, wherein the dust mite allergen is Der p 5 allergen.

4. The process of claim 1, wherein the dust mite further comprises an endoplasmic reticulum (ER) retention signal peptide.

5. The process of claim 1, wherein the vector is modified plant virus vector.

6. The process of claim 5, wherein the vector is selected from the group consisting of Zucchini yellow mosaic virus (ZYMV) and Tobacco mosaic virus (TMV).

7. The process of claim 1, wherein the plant-specific promoter is cauliflower mosaic virus 35S promoter.

8. The process of claim 1, wherein the vector further comprises a selectable marker gene.

9. The process of claim 1, wherein at least one portion of the plant is edible.

10. The process of claim 1, wherein the plant is selected from the group consisting of tobacco, potato and zucchini squash, tomato, lettuce, white grape, banana, rice, radish, carrot, apple, soybean, corn, and berries.

11. The process of claim 10, wherein the plant is selected from the group consisting of Kennebec variety of potato, Nicotina benthamiana and Cucurbia pepo L. var. Zucchini.

12. The process of claim 1, wherein the plant cell or tissue is transformed in step (b) by Agrobacterium-mediated gene transferring, direct DNA uptaking or plant virus infecting step.

13. The process of claim 1 further comprising a step of regenerating a transgenic plant from the plant cell or tissue of step (b) before step (c).

14. The process of claim 1, wherein the dust mite allergen is provided in the form of the transgenic plant itself, a part of the plant, fruit, leaves stems, tubers, seed or extract thereof.

15. A process for producing an antigenic composition, the antigenic composition comprising an dust mite allergen, wherein the dust mite allergen is prepared by a process comprising the steps of:
(a) constructing a vector for plant transformation that comprises a DNA sequence encoding the dust mite allergen operably linked to a plant-specific promoter;
(b) transforming a plant cell or tissue with the vector of step (a); and
(c) obtaining the dust mite allergen from the plant cell or tissue of step (b).

16. The process of claim 15, wherein the dust mite allergen is selected from the group consisting of Der p 5 and Der p 2 allergens.

17. The process of claim 15, wherein the dust mite allergen is Der p 5 allergen.

18. The process of claim 15, wherein the dust mite further comprises an endoplasmic reticulum (ER) retention signal peptide.

19. The process of claim 15, wherein the vector is modified plant virus vector.

20. The process of claim 19, wherein the vector is selected from the group consisting of Zucchini yellow mosaic virus (ZYMV) and Tobacco mosaic virus (TMV).

21. The process of claim 15, wherein the plant-specific promoter is cauliflower mosaic virus 35S promoter.

22. The process of claim 15, wherein the vector further comprises a selectable marker gene.

23. The process of claim 15, wherein at least one portion of the plant is edible.

24. The process of claim 15, wherein the plant is selected form the group consisting of tobacco, potato and zucchini squash, tomato, lettuce, white grape, banana, rice, radish, carrot, apple, soybean, corn, and berries.

25. The process of claim 24, wherein the plant is selected from the group consisting of Kennebec variety of potato, Nicotina benthamiana and Cucurbia pepo L. var. Zucchini.

26. The process of claim 15, wherein the plant cell or tissue is transformed in step (b) by Agrobacterium-mediated gene transferring, direct DNA uptaking or plant virus infecting.

27. The process of claim 15 further comprising a step of regenerating a transgenic plant from the plant cell or tissue of step (b) before step (c).

28. The process of claim 15, wherein the dust mite allergen is provided in the form of the transgenic plant itself, a part of the plant, fruit, leaves, stems, tubers, seed or extract thereof.

29. An antigenic composition comprising unpurified or partial purified recombinant dust mite allergen expressed in a plant at a level sufficient to induce an immunogenic response.

30. The composition of claim 29, wherein the dust mite allergen is selected from the group consisting of Der p 5 and Der p 2allergens.

31. The composition of claim 29, wherein the dust mite allergen is Der p 5 allergen.

32. The composition of claim 29, wherein the plant is selected from the group consisting of tobacco, potato and zucchini squash, tomato, lettuce, white grape, banana, rice, radish, carrot, apple, soybean, corn, and berries.

33. The composition of claim 32, wherein the plant is selected from the group consisting of Kennebec variety of potato, Nicotina benthamiana and Cucurbia pepo L. var. Zucchini.

34. The composition of claim 29, which is for oral administration.
